(19) United States
(12) Patent Application Publication
(10) Pub. No.: US 2022/0125748 A1
(43) Pub. Date: Apr. 28, 2022

(54) HISTONE ACETYLTRANSFERASE (HAT) REGULATORS AND USES THEREOF

(71) Applicant: The Trustees of Columbia University in the City of New York, New York, NY (US)

(72) Inventors: Ottavio ARANCIO, New York, NY (US); Donald W. LANDRY, New York, NY (US); Shixian DENG, White Plains, NY (US); Jennifer Effie AMENGUAL, Scarsdale, NY (US); Elisa ZUCCARELLO, New York, NY (US); Jole FIORITO, Floral Park, NY (US); Yuxuan LIU, Fort Lee, NJ (US); Elisa CALCAGNO, New York, NY (US); Luuk Elard DE VRIES, Amsterdam (NL)

(21) Appl. No.: 17/429,048

(22) PCT Filed: Feb. 7, 2020
(86) PCT No.: PCT/US20/17263

§ 371 Date: Aug. 6, 2021

Related U.S. Application Data
(60) Provisional application No. 62/803,195, filed on Feb. 8, 2019.

(51) Int. Cl. A61K 31/167; A61K 38/15; C07C 235/56; A61P 25/28

(52) U.S. Cl. CPC A61K 31/167; A61P 25/28; C07C 235/56; A61K 38/15

(57) ABSTRACT

The invention provides for compounds that are HAT activators or inhibitors. The invention further provides a method for treating neurodegenerative diseases, cancer and other malignant conditions, or to increase memory in a subject not suffering from a neurodegenerative disease by administering HAT activators or inhibitors to a subject in need thereof. The method further comprises co-administration of HD AC inhibitors with HAT activators or HD AC activators with HAT inhibitors.

### CROSS REFERENCE TO RELATED APPLICATION

This application claims the benefit of U.S. Provisional Patent Application No. 62/803,195, filed on Feb. 8, 2019, entitled “Histone Acetyltransferase (HAT) Regulators and Uses Thereof,” which is incorporated herein by reference.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND

Modulation of the acetylation state of histones, transcription factors, and other regulatory proteins is known to influence their cellular activity. The acetylation state of a protein is governed by the competing activities of two classes of enzymes: histone acetyltransferases (HATs) and histone deacetylases (HDACs). HATs transfer acetyl groups to proteins of interest, while HDACs remove them. Acetylation status influences chromatin condensation and, subsequently, transcription. Deacetylated histones maintain condensed chromatin, which is transcriptionally silent. Acetylated histones lead to open chromatin, which is transcriptionally viable.

Cognitive neurodegenerative disorders are characterized by synaptic dysfunction, cognitive abnormalities, and/or the presence of inclusion bodies through NCS, for example, but not limited to native beta-amyloid, native and phosphorylated tau, native and phosphorylated alpha synuclein, lipofuscin, cleaved TARDBP (TDB-43), oligomeric forms of beta-amyloid, tau and alpha, synuclein, in various percentages and in relation to the specific disease.

Alzheimer's disease (AD is a neurodegenerative disorder characterized by memory loss, synaptic dysfunction and accumulation of amyloid-βpeptides (Aβ). It is caused in part by increased levels of Aβ1-42, and tau. Although AD was described almost a century ago, the molecular mechanisms that lead to its development are still unknown. From a neuropathological point of view, it is characterized by the presence of amyloid plaques and neurofibrillary tangles associated with neuronal degeneration, whereas the clinical hallmark is a progressive memory loss associated with a number of neuropsychiatric symptoms.

Basal HAT activity is essential for normal cellular function. However, hypofunctional, hyperfunctional or dysregulated HAT activity is associated with various acquired and inherited pathological conditions. In a non-limiting example, monoallelic inactivating mutations in the HAT-encoding genes CREBBP and EP300 are linked to altered expression levels of p53 and Bcl6 in cancer (Nature, 2011. 471(7337): p. 189-95; hereby incorporated by reference in its entirety). This is exacerbated in the presence of normally functioning HDACs. In a non-limiting example, these mutations are present in approximately 40% of cases of germinal center-type diffuse large B-cell lymphoma (DLBCL).

Inhibition of HDACs has been widely explored as a potential therapeutic approach for various pathological conditions (Cold Spring Harb Perspect Med, 2016. 6(10): p. a026831; Trends Neurosci, 2009. 32(11): 591-601; Mol Med, 2011. 17(5-6): p. 333-52, each hereby incorporated by reference in its entirety). Therapeutic HAT activators have been reported, but many have poor solubility, poor membrane permeability or unfavorable pharmacological properties. Representative examples include the anacardic acid derivative CTPB and nemorosone (J Biol Chem, 2003. 278(21): p. 19134-40; Chembiochem, 2010. 11(6): p. 818-27; each hereby incorporated by reference in its entirety).

There is an unmet need for bioavailable, pharmacokinetically favorable compounds capable of regulating HAT activity. There is also the need for methods to apply said compounds in the treatment of pathological conditions linked to dysregulated HAT activity. There is further need for said compounds and methods capable of being combined with compounds or methods featuring HDAC regulators, toward generating a synergistic therapeutic effect in pathological conditions linked to dysregulated HAT activity.

### SUMMARY OF THE INVENTION

In one aspect, the invention is directed to a compound of formula (1),

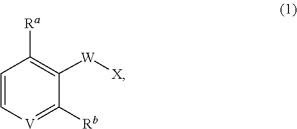

wherein
X is alkyl,

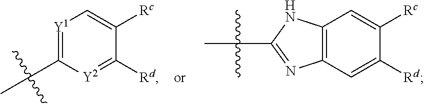

V, Y¹ and Y² are independently —CH— or —N—;
W is —CH₂N(R^{e})—, or —C(O)N(R^{f})—; or
W and R^{b} together with the atoms to which they are bound form a structure of formula Z,
where Z is

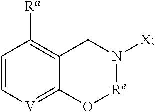

R^{a} is —H, —OH, —O—(C₁-C₆)-alkyl, —O—(C₂-C₆)-heteroalkyl or —O—(C₂-C₆)—N(R^{f}R^{g});
R^{b} is —H, -halo, —(C₂-C₆)-heteroalkyl, —OH, —O—(C₁-C₆)-alkyl, —O—(C₂-C₆)-heteroalkyl, —N—(C₂-C₆)-heteroalkyl, —O—(C₂-C₆)—O-alkyl, —O—(C₂-C₆)—N(R^{f}R^{g}) or N(R^{f})—(C₂-C₆)—N(R^{f}R^{g});
R^{c} and R^{d} are independently —H, -halo or -haloalkyl;
R^{e} is —CH₂— or —C(O)—; and
R^{f} and R^{g} are independently —H, —(C₁-C₆)-alkyl or —(C₂-C₆)-heteroalkyl; or a pharmaceutically acceptable salt thereof.

In some embodiments, the compound of formula (1) is

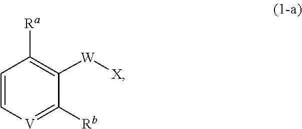

wherein
X is alkyl,

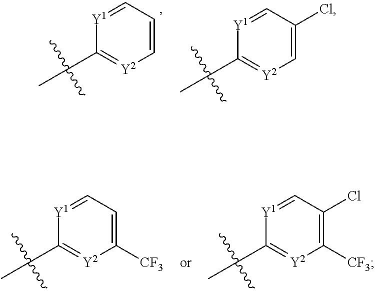

V, Y¹ and Y² are independently —CH— and —N—;

### W is —C(O)N(R^{f})—;

R^{a} is —H, —OH, —O—(C₁-C₆)-alkyl, —O—(C₂-C₆)-heteroalkyl or —O—(C₂-C₆)—N(R^{f}R^{g});
R^{b} is —H, -halo, —(C₂-C₆)-heteroalkyl, —OH, —O—(C₁-C₆)-alkyl, —O—(C₂-C₆)-heteroalkyl, —N—(C₂-C₆)-heteroalkyl, —O—(C₂-C₆)—O-alkyl, —O—(C₂-C₆)—N(R^{f}R^{g}) or N(R^{f})—(C₂-C₆)—N(R^{f}R^{g}); and
R^{f} and R^{g} are independently —H, —(C₁-C₆)-alkyl or —(C₂-C₆)-heteroalkyl; or a pharmaceutically acceptable salt thereof.

In some embodiments, the compound of formula (1) is

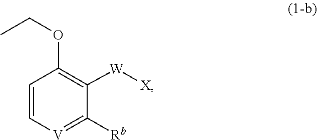

wherein

### X is

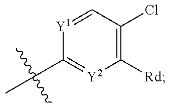

V, Y¹ and Y² are independently —CH— or —N—, wherein at least one of Y¹ and Y² is —N—;

### W is —C(O)N(R^{f})—;

R^{b} is -halo, —O—(C₁-C₂)-alkyl, —O—(C₄-C₆)-alkyl, —O—(C₂-C₆)—OH, —O—(C₂-C₆)—O—(C₁-C₆)-alkyl, —O—(C₂-C₆)—NH₂, —O—(C₂-C₆)—NH(C₁-C₆-alkyl), —O—(C₄-C₆)—N(C₁-C₆-alkyl)₂ or —N(C₁-C₆-alkyl)-(C₂-C₆)-alkyl-N(C₁-C₆-alkyl)₂; and
R^{f} is —H or —(C₂-C₆)-alkyl-N(C₁-C₆-alkyl)₂; or a pharmaceutically acceptable salt thereof.

In some embodiments, the compound of formula (1) is,

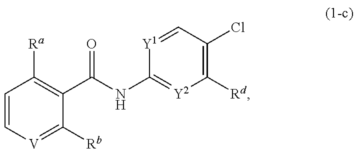

wherein
V, Y¹ and Y² are independently —CH— or —N—;
R^{a} is —H, —OH, —O-methyl, O—(C₃-C₆)-alkyl or —O—(C₂-C₆)—N(C₁-C₆-alkyl)₂; and
R^{b} is halo, —OH, —O—(C₁-C₆)-alkyl or —O—(C₃-C₆)—N(C₁-C₆-alkyl)₂ or —N(R^{f})—(C₃-C₆)-alkyl-N(C₁-C₆-alkyl); and
R^{f} is independently —H, —(C₁-C₆)-alkyl or —(C₂-C₆)-heteroalkyl; or a pharmaceutically acceptable salt thereof.

In some embodiments the compound of formula (1) is

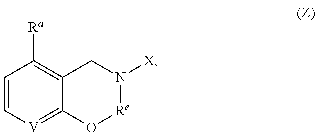

wherein

### X is

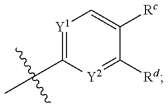

V, Y¹ and Y² are independently —CH— or —N—;
R^{a} is —H, —OH, —O-methyl, —O—(C₃-C₆)-alkyl, —O—(C₂-C₆)-heteroalkyl or —O—(C₂-C₆)—N(R^{f}R^{g});
R^{c} and R^{d} are independently —H, -halo or -haloalkyl; and
R^{e} is —CH₂— or —C(O)—; and
R^{f} and R^{g} are independently —H, —(C₁-C₆)-alkyl or —(C₂-C₆)-heteroalkyl; or a pharmaceutically acceptable salt thereof.

In some embodiments the compound of formula (1) is

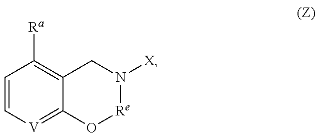

wherein

### X is

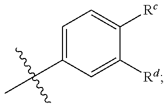

### V is —CH— or —N—;

R^{a} is —H, —OH, —O-methyl, —O—(C₃-C₆)-alkyl, —O—(C₂-C₆)-heteroalkyl or —O—(C₂-C₆)—N(R^{f}R^{g});
R^{c} and R^{d} are independently —H, -halo or -haloalkyl; and
R^{e} is —CH₂— or —C(O)—; and
R^{f} and R^{g} are independently —H, —(C₁-C₆)-alkyl or —(C₂-C₆)-heteroalkyl; or a pharmaceutically acceptable salt thereof.

In another aspect, the compound of formula (1) is capable of regulating HAT activity. In some embodiments, the compound of formula (1) is a HAT activator. In some embodiments, the compound of formula (1) is a HAT inhibitor.

In another aspect, a method is provided for treating inherited and acquired forms of cancer, neurodegenerative diseases, genetic abnormalities, inflammatory diseases, metabolic diseases, lymphatic diseases, renal diseases, cardiac diseases and arterial diseases, representative examples of which appear herein, comprising administering a compound of formula (1) to a subject in need thereof.

In some embodiments, the subject has at least one mutant HAT enzyme gene. In some embodiments, the HAT enzyme mutation is a monoallelic mutation on the EP300 gene. In some embodiments, the HAT enzyme mutation is a monoallelic mutation on the CREBBP gene.

In some embodiments, the HAT regulator is co-administered with a HDAC inhibitor. In some embodiments, a HAT activator is co-administered with a HDAC inhibitor. In some embodiments, a HAT inhibitor is co-administered with a HDAC inhibitor.

### BRIEF DESCRIPTION OF THE FIGURES

FIG. 1 shows chemical structures of representative HAT modulator compounds.

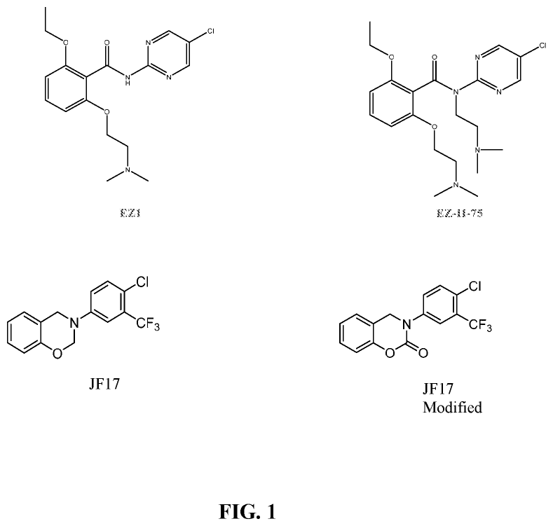

FIG. 2 shows scheme of synthesis of EZ1 and EZ-II-75.

FIG. 3A and FIG. 3B show graphs of the average values of lysine residue acetylation and standard error ranges for EZ1.

FIG. 4A and FIG. 4B show graphs of the average values of lysine residue acetylation and standard error ranges for EZ-II-75.

FIG. 5A and FIG. 5B show graphs of the average values of lysine residue acetylation and standard error ranges for JF2:

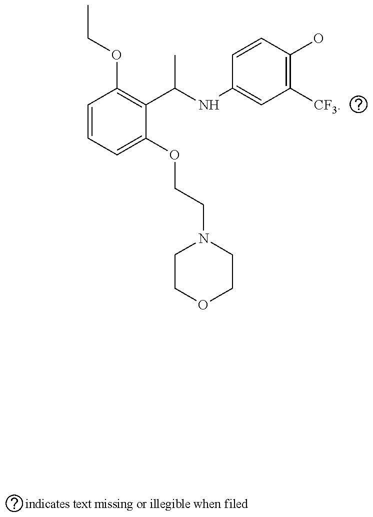 [SMILES: CCOc1cccc(OCCN2CCOCC2)c1C(C)Nc1ccc(O)c(C(F)(F)F)c1]

FIG. 6 shows a graph of the average values of lysine residue acetylation and standard error ranges for JF17:

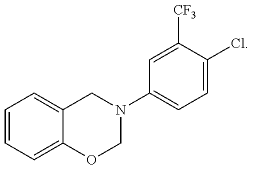 [SMILES: FC(F)(F)c1cc(N2COc3ccccc3C2)ccc1Cl]

FIG. 7A and FIG. 7B show graphs of the average values of lysine residue acetylation and standard error ranges for JF19:

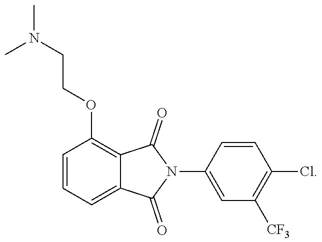 [SMILES: CN(C)CCOc1cccc2c1C(=O)N(c1ccc(Cl)c(C(F)(F)F)c1)C2=O]

FIG. 8 is a graph showing that YF2 rescues oligomeric-Tau (oTau)-induced LTP deficits.

FIG. 9 is a graph showing that YF2 rescues oTau-induced defects in the 2 day radial arm water maze test of spatial short-term memory.

FIG. 10 is a graph showing that YF2 rescues oTau-induced defects in contextual fear memory.

FIG. 11 shows a graph with the average freezing in cued fear associative memory test in the presence oTau with and without YF2.

FIG. 12A and FIG. 12B show graphs with the average time and speed to reach a platform located above the surface of the water in the presence oTau with and without YF2.

FIG. 13A and FIG. 13B show the performance of mice in the open field test in the presence oTau with and without YF2. Both the time spent in the center of the arena and the number of entries in the center are plot.

FIG. 14 shows that the sensory threshold is not affected by the presence oTau with and without YF2.

### DETAILED DESCRIPTION OF THE INVENTION

In one aspect, the invention is directed to a compound of formula (1),

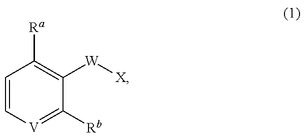

wherein
X is alkyl,

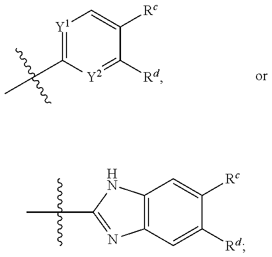

V, Y¹ and Y² are independently —CH— or —N—;
W is —CH₂N(R^{e})—, or —C(O)N(R^{f})—; or
W and R^{b} together with the atoms to which they are bound form a structure of formula Z,
where Z is

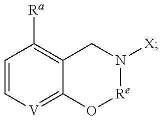

R^{a} is —H, —OH, —O—(C₁-C₆)-alkyl, —O—(C₂-C₆)-heteroalkyl or —O—(C₂-C₆)—N(R^{f}R^{g});
R^{b} is —H, -halo, —(C₂-C₆)-heteroalkyl, —OH, —O—(C₁-C₆)-alkyl, —O—(C₂-C₆)-heteroalkyl, —N—(C₂-C₆)-heteroalkyl, —O—(C₂-C₆)—O-alkyl, —O—(C₂-C₆)—N(R^{f}R^{g}) or N(R^{f})—(C₂-C₆)—N(R^{f}R^{g});
R^{c} and R^{d} are independently —H, -halo or -haloalkyl;
R^{e} is —CH₂— or —C(O)—; and
R^{f} and R^{g} are independently —H, —(C₁-C₆)-alkyl or —(C₂-C₆)-heteroalkyl; or a pharmaceutically acceptable salt thereof.

In some embodiments, both of Y¹ and Y² are —N—.

In some embodiments, only one of Y¹ and Y² is —N— and when X is

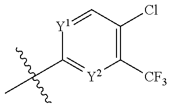

and R^{b} is —O(CH₂)₂—N(CH₃)₂, then R^{a} is —H, —OH, —O-methyl, O—(C₃-C₆)-alkyl or —O—(C₂-C₆)—N(C₁-C₆-alkyl)₂ or a pharmaceutically acceptable salt thereof.

In accordance with other embodiments, W and R^{b} together with the atoms to which they are bound form the structure of formula Z, where Z is

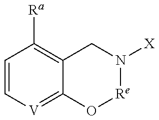

and when X is

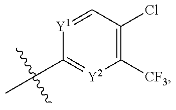

then R^{a} is —H, —OH, —O-methyl, O—(C₃-C₆)-alkyl or —O—(C₂-C₆)—N(C₁-C₆-alkyl)₂ or a pharmaceutically acceptable salt thereof.

In some embodiments, wherein X is

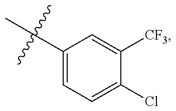 [SMILES: *c1ccc(Cl)c(C(F)(F)F)c1]

W is —C(O)NH—, V is —CH— and R^{a} is H, R^{b} is not —O(CH₂)₂N(CH₃)₂ or —NH(CH₂)₂N(CH₃)₂. In some embodiments wherein X is

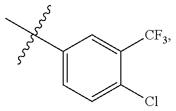 [SMILES: *c1ccc(Cl)c(C(F)(F)F)c1]

W is —C(O)NH—, V is —CH— and R^{b} is H, R^{a} is not —O-n-propyl, —O-n-butyl or —O-n-hexyl.

In some embodiments wherein X is

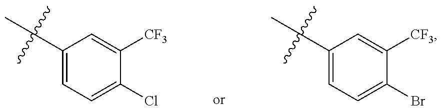 [SMILES: *c1ccc(Cl)c(C(F)(F)F)c1 ; "or" ; *c1ccc(Br)c(C(F)(F)F)c1]

W is —C(O)NH—, V is —CH— and R^{b} is H, R^{a} is not —OH, —O-n-propyl or —O-n-butyl.

In some embodiments wherein X is

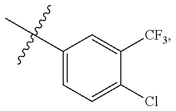 [SMILES: *c1ccc(Cl)c(C(F)(F)F)c1]

W is —CH₂NH—, V is —CH— and R^{a}

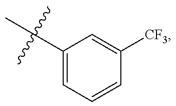 [SMILES: *c1cccc(C(F)(F)F)c1]

is —O-ethyl, R^{b} is not —OH. In some embodiments wherein X is V is —CH—, R^{a} is O-ethyl, and W is —C(O)—NH—, R^{b} is not —O(CH₂)₂—OH. In some embodiments wherein X is

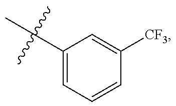 [SMILES: *c1cccc(C(F)(F)F)c1]

V is —CH—, R^{a} is O-ethyl, and W is —C(O)—NH—, R^{b} is not —O(CH₂)₂—N(CH₃)₂.

In some embodiments wherein formula (1) is structure Z, wherein X is

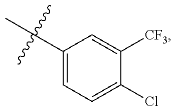 [SMILES: *c1ccc(Cl)c(C(F)(F)F)c1]

V is —CH— and R^{e} is —CH₂—, R^{a} is not —O-ethyl.

In some embodiments, X is alkyl,

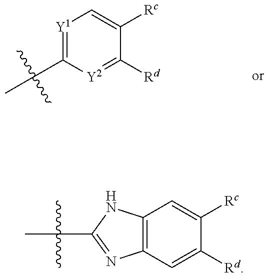

In some embodiments, X is (C₁-C₆)-alkyl,

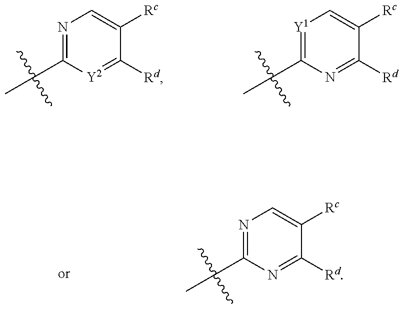

In some embodiments, X is methyl,

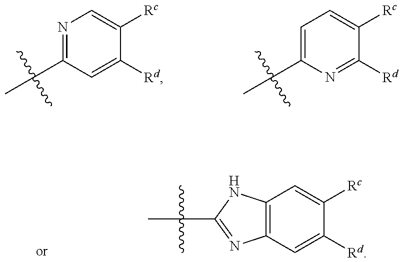

In some embodiments, W is —C(O)N(R^{f})—, wherein R^{f} is —H, —(C₁-C₆)-alkyl or —(C₂-C₆)-heteroalkyl. In some embodiments, R^{f} is —(CH₂)₂N(CH₃)₂.

In some embodiments, W is —CH₂N(R^{e})—, where W and R^{b} together with the atoms to which they are bound form

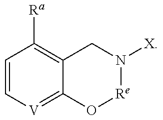

In some embodiments, X is

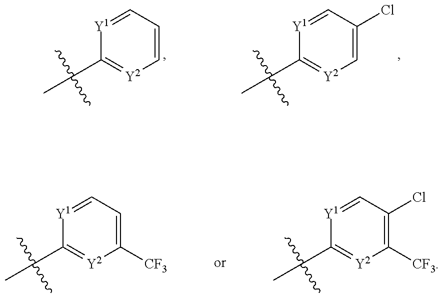

In some embodiments, R^{e} is —CH₂— or —C(O)—.

In some embodiments, V, Y¹ and Y² are independently —CH— or —N—. In some embodiments, V is —CH— when at least one of Y¹ and Y² is —N—. In some embodiments, V is —CH— and both Y¹ and Y² are —N—. In some embodiments, V is —N— when at least one of Y¹ and Y² is —CH—. In some embodiments, V, Y¹ and Y² are —CH—. In some embodiments, V, Y¹ and Y² are —N—.

In some embodiments, R^{c} and R^{d} are independently —H, -halo, -haloalkyl. In some embodiments, R^{c} is —H when R^{d} is -halo or -haloalkyl. In some embodiments, R^{c} is —H when R^{d} is -halo. In some embodiments, R^{c} is H when R^{d} is —Cl. In some embodiments, R^{c} is -halo or -haloalkyl when R^{d} is H. In some embodiments, R^{c} is -haloalkyl when R^{d} is H. In some embodiments, R^{c} is —CF₃ when R^{d} is H. In some embodiments, R^{c} is -halo when R^{d} is -haloalkyl. In some embodiments, R^{c} is -haloalkyl when R^{d} is -halo. In some embodiments, R^{c} is —Cl when R^{d} is -haloalkyl. In some embodiments, R^{c} is —CF₃ when R^{d} is halo. In some embodiments, R^{c} is —Cl when R^{d} is —CF₃. In some embodiments, R^{c} is —CF₃ when R^{d} is —Cl. In some embodiments, R^{c} and R^{d} are H.

In some embodiments, R^{a} is —H, —OH, —O—(C₁-C₆)-alkyl, —O—(C₂-C₆)-heteroalkyl or —O—(C₂-C₆)—N(R^{f}R^{g}), wherein R^{f} and R^{g} are independently —H, —(C₁-C₆)-alkyl or —(C₂-C₆)-heteroalkyl. In some embodiments, R^{a} is —H, —OH, —O—(C₁-C₆)-alkyl, —O—(C₂-C₆)-heteroalkyl or —O—(C₂-C₆)—N(R^{f})₂, wherein R^{f} is —H, —(C₁-C₆)-alkyl or —(C₂-C₆)-heteroalkyl. In some embodiments, R^{a} is —H, —OH, —O—(C₁-C₃)-alkyl, —O—(C₂)-heteroalkyl or —O—(C₂)—N(R^{f})₂. In some embodiments, R^{a} is —H, —OH, —O-methyl, —O-ethyl, —O-n-propyl, —O-isopropyl or —O—(CH₂)₂—N(R^{f})₂. In some embodiments, R^{a} is —H, —OH, —O-methyl, —O-ethyl, —O-n-propyl, —O-isopropyl or —O—(CH₂)₂—N(CH₃)₂.

In some embodiments, R^{b} is —H, -halo, —(C₂-C₆)-heteroalkyl, —OH, —O—(C₁-C₆)-alkyl, —O—(C₂-C₆)-heteroalkyl, —N—(C₂-C₆)-heteroalkyl, —O—(C₂-C₆)—O-alkyl, —O—(C₂-C₆)—N(R^{f}R^{g}) or N(R^{f})—(C₂-C₆)—N(R^{f}R^{g}), wherein R^{f} and R^{g} are independently —H, —(C₁-C₆)-alkyl or —(C₂-C₆)-heteroalkyl. In some embodiments, R^{b} is —H, -halo, —(C₂-C₆)-heteroalkyl, —OH, —O—(C₁-C₆)-alkyl, —O—(C₂-C₆)-heteroalkyl, —N—(C₂-C₆)-heteroalkyl, —O—(C₂-C₆)—O-alkyl, —O—(C₂-C₆)—N(R^{f}R^{g}) or N(R^{f})—(C₂-C₆)—N(R^{f})₂. In some embodiments, R^{b} is -halo, —(C₃)-heteroalkyl, —OH, —O—(C₁-C₃)-alkyl, —O—(C₂)-heteroalkyl, —N—(C₂)-heteroalkyl, —O—(C₂)—O-alkyl, —O—(C₂)—N(R^{f}R^{g}) or N(R^{f})—(C₂)—N(R^{f})₂. In some embodiments, R^{b} is —Br, —F, —(CH₂)₃—N(R^{f})₂, —OH, —O-methyl, —O-ethyl, —O-n-propyl, —O—(CH₂)₂—OH, —O—(CH₂)₂—O-alkyl, —O—(CH₂)₂—N(R^{f}R^{g}) or N(R^{f})—(CH₂)₂—N(R^{f})₂. In some embodiments, R^{f} is H or CH₃. In some embodiments, R^{b} is —Br, —F, —(CH₂)₃—N(CH₃)₂, —OH, —O-methyl, —O-ethyl, —O-n-propyl, —O—(CH₂)₂—OCH₃, —O—(CH₂)₂—NH₂, —O—(CH₂)₂—NHCH₃, —O—(CH₂)₂—N(CH₃)₂ or N(CH₃)—(CH₂)₂—N(CH₃)₂.

In some embodiments, the compound of formula (1) is

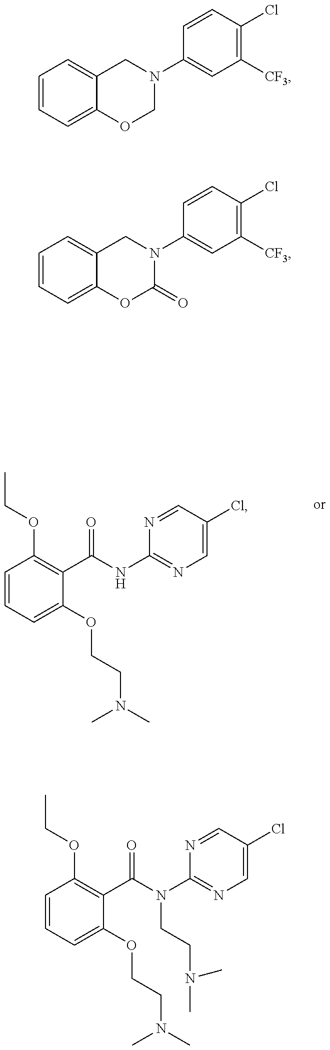 [SMILES: FC(F)(F)c1cc(N2COc3ccccc3C2)ccc1Cl ; O=C1Oc2ccccc2CN1c1ccc(Cl)c(C(F)(F)F)c1 ; CCOc1cccc(OCCN(C)C)c1C(=O)Nc1ncc(Cl)cn1 ; "or" ; CCOc1cccc(OCCN(C)C)c1C(=O)N(CCN(C)C)c1ncc(Cl)cn1]

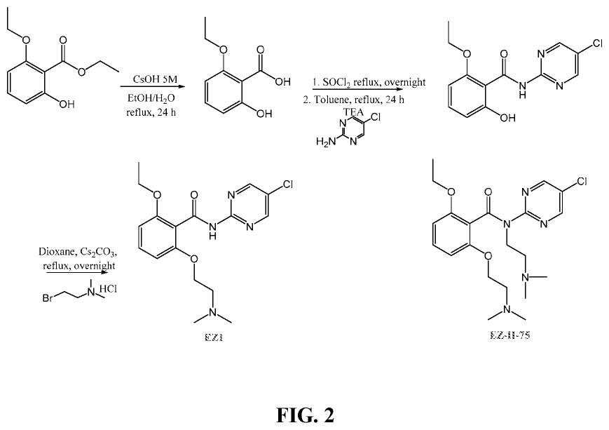

or a pharmaceutically acceptable salt thereof. Representative schemes of synthesis are provided in FIG. 2. Other representative schemes for producing the disclosed compounds are described in WO/2011/072243, WO 2012/08420, WO 2012/171008, WO 2015/153410 and WO 2018/017858 (each of which is hereby incorporated by reference in its entirety).

In another aspect, the compounds comprising formula (1) are capable of regulating HAT activity. In some embodiments, the HAT regulator is a HAT activator. In some embodiments, the HAT regulator is a HAT inhibitor.

In some embodiments, the HAT activator is

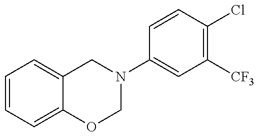 [SMILES: FC(F)(F)c1cc(N2COc3ccccc3C2)ccc1Cl]

or a pharmaceutically acceptable salt thereof.

In some embodiments, the HAT is a type A or type B HAT. In some embodiments, the HAT is HAT1, GCN5, PCAF, ATF-2, Tip60, MOZ, MORF, HBO1, MOF, p300, CBP, ACTR/SRC-1 or CLOCK. In some embodiments, the type A HAT is HAT1, GCN5, PCAF, ATF-2, Tip60, MOZ, MORF, HBO1, MOF, p300, CBP, ACTR/SRC-1 or CLOCK. In some embodiments, the type B HAT is HAT1. In some embodiments, the type A HAT belongs to a HAT family. In some embodiments, the HAT family is GNAT, MYST, nuclear receptor coactivators or P300/CBP. In some embodiments, the GNAT family includes HAT1, GCN5, PCAF or ATF-2. In some embodiments, the MYST family includes Tip60, MOZ, MORF, HBO1 or MOF. In some embodiments, the P300/CBP family includes P300 and CBP. In some embodiments, the nuclear receptor coactivators family includes ACTR/SRC-1 and CLOCK. In some embodiments, the type A HAT does not belong to an established HAT family.

In another aspect, a method is provided for treating neurodegenerative diseases, inherited and acquired forms of cancer, genetic abnormalities, inflammatory diseases, metabolic diseases, lymphatic diseases, renal diseases, cardiac diseases and arterial diseases, representative examples of which appear herein, comprising administering a compound of formula (1) to a subject in need thereof.

In some embodiments, a method for treating neurodegenerative diseases is provided, comprising administering a compound of formula (1) to a subject in need thereof. Representative neurodegenerative diseases include, but are not limited to, adrenoleukodystrophy (ALD), Alexander's disease, Alpers' disease, Alzheimer's disease, corticobasal degeneration (CBD), argyrophilic grain disease (AGD), and globular glial tauopathy (GGT), the neurofibrillary tangle-predominant senile dementia (now included also in the category of primary age-related tauopathy, PART), behavioral variant frontotemporal dementia; Semantic variant primary progressive aphasia, non-fluent/agrammatic variant primary progressive aphasia, logopenic variant primary progressive aphasia, Rubinstein-Taybi syndrome, amyotrophic lateral sclerosis (Lou Gehrig's disease), ataxia telangiectasia, batten disease (also known as Spielmeyer-Vogt-Sjögren-Batten disease), bovine spongiform encephalopathy (BSE), canavan disease, cockayne syndrome, corticobasal degeneration, Creutzfeldt-Jakob disease, familial fatal insomnia, frontotemporal lobar degeneration, Huntington's disease, HIV-associated dementia, Kennedy's disease, Krabbe's disease, Lewy body dementia, neuroborreliosis, Machado-Joseph disease (spinocerebellar ataxia type 3), multiple system atrophy, multiple sclerosis, narcolepsy, Niemann Pick disease, Parkinson's disease, Pelizaeus-Merzbacher disease, Pick's disease, primary lateral sclerosis, Prion diseases, progressive supranuclear palsy, Refsum disease, Sandhoff disease, Schilder's disease, subacute combined degeneration of spinal cord secondary to pernicious anemia, Spielmeyer-Vogt-Sjogren-Batten disease (also known as Batten disease), spinocerebellar ataxia (multiple types with varying characteristics), spinal muscular atrophy, Steele-Richardson-Olszewski disease, Tabes dorsalis and toxic encephalopathy. In some embodiments, a method for preventing, restoring or otherwise improving motor skills, learning, memory or cognition in said subject with a neurodegenerative disease is provided, comprising administration of a compound of formula (1) in a subject in need thereof. In some embodiments, subject does not have a neurodegenerative disease. Other methods of treating neurodegenerative diseases with HAT regulators are disclosed, for example, in US Patent Publications Nos. 2013/0121919, 2018/0244603, 2018/0050982, 2018/0360842 or 2018/0021273 (each of which is hereby incorporated by reference in its entirety).

In some embodiments, a method for treating cancer is provided, comprising administering a compound of formula (1) to a subject in need thereof. Representative types of cancer include, but are not limited to, B cell lymphoma, diffuse large B-cell lymphoma (DLBCL), colon cancer, lung cancer, non-small cell lung cancer (SCLC), renal cancer, bladder cancer, peripheral T cell lymphoma (PTCL-NOS), NK/T cell lymphoma (NKTCL), follicular lymphoma, myeloma, leukemia, chronic myeloid leukemia, acute myeloid leukemia, chronic lymphocytic leukemia, acute lymphocytic leukemia, hematopoietic neoplasias, thymoma, lymphoma, sarcoma, lung cancer, liver cancer, non-Hodgkin's lymphoma, Hodgkin's lymphoma, uterine cancer, renal cell carcinoma, hepatoma, adenocarcinoma, breast cancer, pancreatic cancer, liver cancer, prostate cancer, head and neck carcinoma, thyroid carcinoma, soft tissue sarcoma, ovarian cancer, primary or metastatic melanoma, squamous cell carcinoma, basal cell carcinoma, brain cancer, angiosarcoma, hemangiosarcoma, bone sarcoma, fibrosarcoma, myxosarcoma, liposarcoma, chondrosarcoma, osteogenic sarcoma, chordoma, angiosarcoma, endotheliosarcoma, lymphangiosarcoma, lymphangioendothelioma, synovioma, testicular cancer, uterine cancer, cervical cancer, gastrointestinal cancer, stomach cancer, esophageal cancer, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, Waldenstrom's macroglobulinemia, papillary adenocarcinomas, cystadenocarcinoma, bronchogenic carcinoma, bile duct carcinoma, choriocarcinoma, seminoma, embryonal carcinoma, Wilms' tumor, lung carcinoma, epithelial carcinoma, cervical cancer, testicular tumor, glioma, astrocytoma, medulloblastoma, craniopharyngioma, ependymoma, pinealoma, hemangioblastoma, acoustic neuroma, oligodendroglioma, meningioma, retinoblastoma, leukemia, melanoma, neuroblastoma, small cell lung carcinoma, bladder carcinoma, lymphoma, multiple myeloma and medullary carcinoma. In some embodiments, DLBCL is germinal center-derived DLBCL, activated B-cell-derived (ABC) DLBCL or non-germinal center DLBCL. In some embodiments, method further comprises regenerating epithelial cells and regulating inflammation. Other methods of treating cancer with HAT regulators are disclosed, for example, in US Patent Publications Nos. 2013/0121919, 2018/0244603 and 2018/0021273 (each of which is hereby incorporated by reference in its entirety).

In some embodiments, a method for treating cancer of the representative types listed above is provided, comprising administering a compound of formula (1) to a subject in need thereof, wherein the subject has at least one mutant HAT enzyme gene. In some embodiments, the HAT enzyme mutation is a monoallelic mutation on the EP300 gene. In some embodiments, the HAT enzyme mutation is a monoallelic mutation on the CREBBP gene.

In some embodiments, a method for treating genetic abnormalities, inflammatory diseases, metabolic diseases, lymphatic diseases, renal diseases, cardiac diseases and arterial diseases is provided, comprising administering a compound of formula (1) to a subject in need thereof. Representative types of disease include, but are not limited to, therapeutic ateriogenesis, Kawasaki disease, Crohn's disease, DiGeorge syndrome, Rubenstein-Taybi syndrome (RTS), cardiac hypertrophy, insulin resistance, diabetes, type 2 diabetes, obesity, lymphoid hyperplasia and chronic kidney disease.

In some embodiments, the present disclosure provides pharmaceutical compositions comprising an effective amount of a compound of Formula (I), or a pharmaceutically acceptable salt thereof. In some embodiments, the pharmaceutical compositions provided herein comprise one or more pharmaceutically acceptable carriers or excipients.

In various embodiments, the pharmaceutical compositions of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

The effective amount of a compound of Formula (I), pharmaceutically acceptable salts, esters, prodrugs, hydrates, solvates and isomers thereof, or a pharmaceutical composition comprising a compound of Formula (I) or a pharmaceutically acceptable salt thereof may be determined by one skilled in the art based on known methods.

In one embodiment, a pharmaceutical composition or a pharmaceutical formulation of the present disclosure comprises a compound of Formula (I) or a pharmaceutically acceptable salt thereof, and a pharmaceutically acceptable carrier, diluent, and/or excipient. Pharmaceutically acceptable carriers, diluents or excipients include without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

In one embodiment, suitable pharmaceutically acceptable carriers include, but are not limited to, inert solid fillers or diluents and sterile aqueous or organic solutions. Pharmaceutically acceptable carriers are well known to those skilled in the art and include, but are not limited to, from about 0.01 to about 0.1 M and preferably 0.05M phosphate buffer or 0.8% saline. Such pharmaceutically acceptable carriers can be aqueous or non-aqueous solutions, suspensions and emulsions. Examples of non-aqueous solvents suitable for use in the present application include, but are not limited to, propylene glycol, polyethylene glycol, vegetable oils such as olive oil, and injectable organic esters such as ethyl oleate.

Aqueous carriers suitable for use in the present application include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the present application can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the present application include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellant.

Solid carriers suitable for use in the present application include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

Parenteral carriers suitable for use in the present application include, but are not limited to, sodium chloride solution, Ringer's dextrose, dextrose and sodium chloride, lactated Ringer's and fixed oils. Intravenous carriers include fluid and nutrient replenishers, electrolyte replenishers such as those based on Ringer's dextrose and the like. Preservatives and other additives can also be present, such as, for example, antimicrobials, antioxidants, chelating agents, inert gases and the like.

Carriers suitable for use in the present application can be mixed as needed with disintegrants, diluents, granulating agents, lubricants, binders and the like using conventional techniques known in the art. The carriers can also be sterilized using methods that do not deleteriously react with the compounds, as is generally known in the art.

Diluents may be added to the formulations of the present invention. Diluents increase the bulk of a solid pharmaceutical composition and/or combination, and may make a pharmaceutical dosage form containing the composition and/or combination easier for the patient and care giver to handle. Diluents for solid compositions and/or combinations include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, and talc.

The pharmaceutical composition of the present invention may be prepared into any type of formulation and drug delivery system by using any of the conventional methods well-known in the art. The inventive pharmaceutical composition may be formulated into injectable formulations, which may be administered by routes including intrathecal, intraventricular, intravenous, intraperitoneal, intranasal, intraocular, intramuscular, subcutaneous or intraosseous. Also, it may also be administered orally, or parenterally through the rectum, the intestines or the mucous membrane in the nasal cavity (see Gennaro, A. R., ed. (1995) Remington's Pharmaceutical Sciences). Preferably, the composition is administered topically, instead of enterally. For instance, the composition may be injected, or delivered via a targeted drug delivery system such as a reservoir formulation or a sustained release formulation.

The pharmaceutical formulation of the present invention may be prepared by any well-known methods in the art, such as mixing, dissolving, granulating, dragee-making, levigating, emulsifying, encapsulating, entrapping, or lyophilizing processes. As mentioned above, the compositions of the present invention may include one or more physiologically acceptable carriers such as excipients and adjuvants that facilitate processing of active molecules into preparations for pharmaceutical use.

Proper formulation is dependent upon the route of administration chosen. For injection, for example, the composition may be formulated in an aqueous solution, preferably in physiologically compatible buffers such as Hank's solution, Ringer's solution, or physiological saline buffer. For transmucosal or nasal administration, penetrants appropriate to the barrier to be permeated are used in the formulation. Such penetrants are generally known in the art. In a one embodiment of the present invention, the inventive compound may be prepared in an oral formulation. For oral administration, the compounds can be formulated readily by combining the active compounds with pharmaceutically acceptable carriers known in the art. Such carriers enable the disclosed compound to be formulated as tablets, pills, dragees, capsules, liquids, gels, syrups, slurries, suspensions and the like, for oral ingestion by a subject. The compounds may also be formulated in rectal compositions such as suppositories or retention enemas, e.g., containing conventional suppository bases such as cocoa butter or other glycerides.

Pharmaceutical preparations for oral use may be obtained as solid excipients, optionally grinding a resulting mixture, and processing the mixture of granules, after adding suitable adjuvants, if desired, to obtain tablets or dragee cores. Suitable excipients may be, in particular, fillers such as sugars, including lactose, sucrose, mannitol, or sorbitol; cellulose formulation such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP) formulation. Also, disintegrating agents may be employed, such as cross-linked polyvinylpyrrolidone, agar, or alginic acid or a salt thereof such as sodium alginate. Also, wetting agents, such as sodium dodecyl sulfate and the like, may be added.

In some embodiments, the HAT regulator is co-administered with a HDAC inhibitor. In some embodiments, a HAT activator is co-administered with a HDAC inhibitor. In some embodiments, a HAT inhibitor is co-administered with a HDAC activator.

In some embodiments, a HAT regulator compound and a HDAC regulator compound are used, formulated for use and/or administered to the subject. In some embodiments, the HAT regulator compound and the HDAC regulator are used, formulated for use and/or administered to the subject at the same time, optionally as a composition comprising the HAT regulator compound and the HDAC regulator, or as two separate doses. In some embodiments, the HAT regulator compound and the HDAC regulator are used, formulated for use and/or administered to the subject at different times. For example, some embodiments, the HAT regulator compound is used or administered prior to, or after the HDAC regulator. In one embodiment, the HAT regulator is used or administered prior to, or after the HDAC regulator separated by a time of at least about 1 minute, 2 minutes, 5 minutes, 10 minutes, 30 minutes: 45 minutes, 1 hour, 1.5 hours, 2 hours, 3 hours, 4 hours, 5 hours, 6 hours, 8 hours, 10 hours, 12 hours 16 hours, or 24 hours. Optionally, in some embodiments the HDAC regulator is used, formulated for use and/or administered to the subject separated by more than about 24 hours, 36 hours, 48 hours, 3 days, 4 days, 5 days, 6 days, or one week.

Pharmaceutically acceptable salts are known in the art, and can be selected from those listed in Berge, et al. [“Pharmaceutical Salts,” J. Pharm. Sci., 66(1):1-19 (January 1977); herein incorporated by reference in its entirety]. In some embodiments, a pharmaceutically acceptable salt of a compound of formula (1) is an acid addition salt, for example a hydrohalide (such as hydrochloride or hydrobromide), sulfate or phosphate salt. In some embodiments, the pharmaceutically acceptable salt is a base addition salt, for example a sodium, potassium, calcium or ammonium salt. In some embodiments, the base addition salt is a tetrafluoroboro salt.

The HAT regulator compound and HDAC regulator compound of the invention can be incorporated into pharmaceutical compositions suitable for administration. Such compositions can comprise a compound of formula (1) and a pharmaceutically acceptable carrier. Such compositions can also comprise these compounds together with a HDAC regulator and a pharmaceutically acceptable carrier. The compositions can be administered alone or in combination with at least one other agent, such as a stabilizing compound, which can be administered in any sterile, biocompatible pharmaceutical carrier including, but not limited to, saline, buffered saline, dextrose and water. The compositions can be administered to a patient alone, or in combination with other agents, drugs or hormones. A pharmaceutically acceptable carrier can comprise any and all solvents, dispersion media, coatings, antibacterial and antifungal agents, isotonic and absorption delaying agents, and the like, compatible with pharmaceutical administration. The use of such media and agents for pharmaceutically active substances is well known in the art.

### EXAMPLES

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1—In Vitro Acetylation Assay

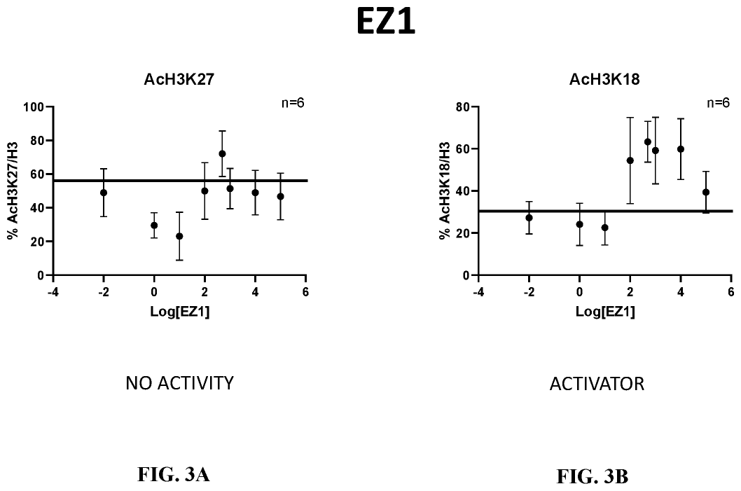

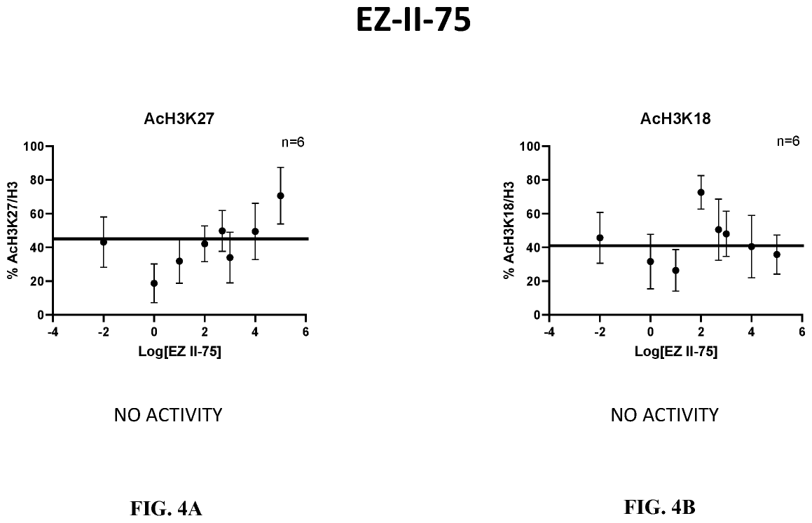

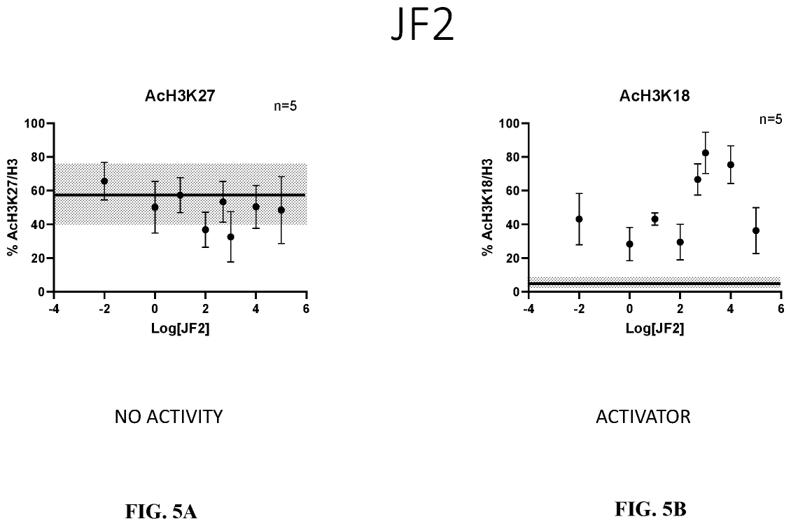

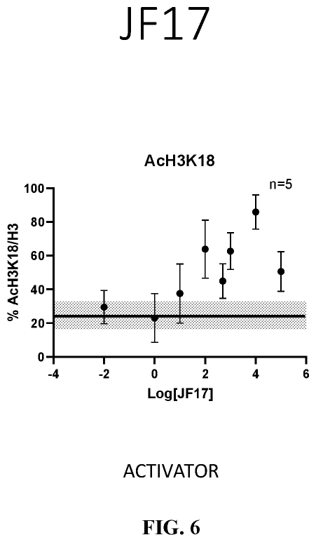

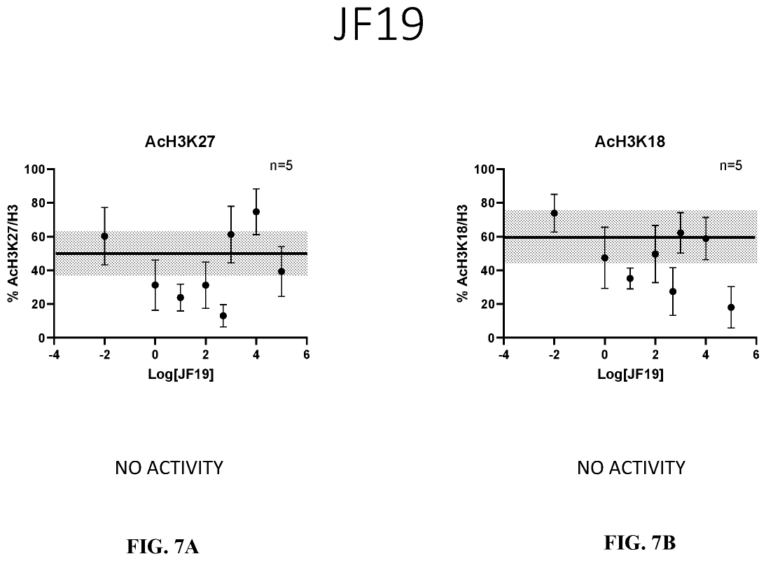

The aim of the in vitro acetylation assay is to measure the enzymatic activity of the various compounds towards p300. Results are provided in FIG. 3A to FIG. 7B for average values of lysine residue acetylation (continuous line) and their standard error range measured in the absence of compound and DMSO, wherein “n” represents the number of replicates.

First, the drug is prepared:

| stock conc. | 10 | mM | 1 | mM | 100 | μM | 50 | μM | 10 | μM | 1 | μM | 0.1 | μM | 0.001 | μM | 0 | | Blank |
|---|---|---|---|---|---|---|---|---|---|---|---|---|---|---|---|---|---|---|---|
| DRUG (μL) | from | | 10 of | | 10 of | | 10 of | | 10 of | | 10 of | | 10 of | | 10 of | | | | |
| | powder | | 10 mM | | 1 mM | | 100 μM | | 50 μM | | 10 μM | | 1 μM | | 0.1 μM | | | | |
| 1% DMSO (μL) | | | 90 | | 90 | | 10 | | 40 | | 90 | | 90 | | 990 | | 10 | | |
| HAT Buffer | 100 | μM | 10 | μM | 1 | μM | 500 | nM | 100 | nM | 10 | nM | 1 | nM | 0.01 | nM | 0 | | Blank |
| final conc. | | | | | | | | | | | | | | | | | | | |
| Sample No. | 1 | | 2 | | 3 | | 4 | | 5 | | 6 | | 7 | | 8 | | 9 | 10 | % of DMSO |
| DMSO stock solutions | 10000 | | 1000 | | 100 | | 50 | | 10 | | 1 | | 0.1 | | 0.001 | | 0 | Blank | 100% |
| (mM) | | | | | | | | | | | | | | | | | | | |
| Vol. (μL) of 5xHAT | 18 | | 18 | | 18 | | 18 | | 18 | | 18 | | 18 | | 18 | | 18 | 18 | |
| assay buffer | | | | | | | | | | | | | | | | | | | |
| Vol. (μL) of DMSO | 2 | | 2 | | 2 | | 2 | | 2 | | 2 | | 2 | | 2 | | 2 | / | 2 μl |
| stock solutions in final | | | | | | | | | | | | | | | | | | | (pure |
| 20 uL of assay buffer | | | | | | | | | | | | | | | | | | | DMSO) |
| 10× dil. stock in assay | 1000 | | 100 | | 10 | | 5 | | 1 | | 0.1 | | 0.01 | | 0.0001 | | 0 | Blank | 10% |
| buffer (mM) | | | | | | | | | | | | | | | | | | | |
| Vol. (μL) of 10× dil. | 2 | | 2 | | 2 | | 2 | | 2 | | 2 | | 2 | | 2 | | 2 | 2 | |
| stock to final 20 ul | | | | | | | | | | | | | | | | | | | |
| Final conc. in assay | 100 | | 10 | | 1 | | 0.5 | | 0.1 | | 0.01 | | 0.001 | | 0.00001 | | 0 | Blank | 1% |
| (mM) | | | | | | | | | | | | | | | | | | | |
| Final conc. in assay | 100 | μM | 10 | μM | 1 | μM | 500 | nM | 100 | nM | 10 | nM | 1 | mM | 0.01 | nM | 0 | Blank | |

(μM)

Second, dilute p300 in AM1 buffer to a concentration of 40 ng/μL (final concentration in the reaction 20 ng/μL). This is accomplished by diluting 1 μL of p300 (at 0.4 μg/μL) into 19 μL of AM1 buffer.

Third, prepare the Master Mix. Prepare the Master Mix in low protein binding tubes (ThermoFisher Cat. No. 90410), 20 μL system.

| Components | 1x | Final conc. in 20 μL |
|---|---|---|
| P300 (20 ng/μL) | 2 μl | |
| 5X HAT buffer | 4 μl | |
| Compound | 2 μl | 0, 0.01, 1, 10, 100, 500, 10³, 10⁴, 10⁵ |

Incubate reactions at 30° C. for 30 minutes.

| | | |
|---|---|---|
| Histone 3.3 (1 μg/μl) | 1 μl | |
| Ac—CoA (0.5 μM) | 1 μl | 0.025 μM |
| H2O | 10 μl | |
| TOTAL | 20 μl | |

Incubate reactions at 30° C. for 1 hour.

Fourth, perform the western blot assay.
Add 6.7 μL of Laemmli Sample Buffer 4× (Bio-Rad Laboratories Cat. No. 161-0737) for each reaction and boil samples at 95° C. for 5 min.
Charge 10 μL for each sample in 2 Tris Glycine 4-15% gels (Bio-Rad Laboratories Cat. No. 456-1086). Running Buffer: Tris-Glycine 1× (pour in the cell up to the writing “2 gels”).
Run at 90 V for 1 hour (until the gel front reaches the green line of cell).
Semi-dry transfer with Trans-Blot® Turbo™ Blotting System from Bio-Rad Laboratories (Trans-Blot® Turbo™ RTA midi PVDF transfer kit Cat. No1704275).
   Activate PVDF membrane in methanol for 5 minutes and then wet in Trans-Blot® Turbo™ transfer buffer.
   Wet stacks in transfer buffer.
Transfer at 23 V 1.3 A for 7 minutes.
Blocking buffer: 5% Non-Fat milk in TBST (tween 0.1%) 1 hour at room temperature.
Cut the membranes to get Ac-His 3 at 17 kDa and p300 at 300 kDa.
Incubate with primary antibodies overnight at 4° C.
The day after, make 3 washes in TBST (Tween 0.1%) of 10 minutes.
Incubate with secondary antibody at room temperature for 1 hour.
Make 3 washes in TBST (Tween 0.1%) of 5 minutes.
Incubate membranes with ECL (SuperSignal West Dura Extended Duration Substrate from ThermoFisher) for 5 minutes at room temperature.
Acquire the image with ChemiDoc Odyssey Fc 2 minutes.

To detect total H3 (loading control), strip the membranes with Restore™ Western Blot Stripping Buffer according to the manufacturer protocol.

| | Primary antibody | Secondary antibody |
|---|---|---|
| | dilution | dilution |
| Mouse p300 | 1:1000 | 1:2000 |
| Rabbit H3K27ac | 1:2000 | 1:2000 |
| Rabbit H3K18ac | 1:2000 | 1:2000 |
| Rabbit H3 | 1:5000 | 1:5000 |

### Example 2—Stability in Human Liver Microsomes

Compounds were tested for stability in human liver microsomes.

Experimental Procedure
Mixed-gender human liver microsomes (Lot #1210347) were purchased from XenoTech. The reaction mixture, minus NADPH, was prepared as described below. The test article was added into the reaction mixture at a final concentration of 1 μM. The control compound, testosterone, was run simultaneously with the test articles in a separate reaction. An aliquot of the reaction mixture (without cofactor) was equilibrated in a shaking water bath at 37° C. for 3 minutes. The reaction was initiated by the addition of the cofactor, and the mixture was incubated in a shaking water bath at 37° C. Aliquots (100 μL) were withdrawn at 0, 10, 20, 30, and 60 minutes. Test article and testosterone samples were immediately combined with 400 μL of ice-cold 50/50 acetonitrile (ACN)/H2O containing 0.1% formic acid and internal standard to terminate the reaction. The samples were then mixed and centrifuged to precipitate proteins. All samples were assayed by LC-MS/MS using electrospray ionization.
Analytical conditions are outlined in Appendix 1. The peak area response ratio (PARR) to internal standard was compared to the PARR at time 0 to determine the percent remaining at each time point. Half-lives and clearance were calculated using GraphPad software, fitting to a single-phase exponential decay equation.

Reaction Composition

Liver Microsomes 0.5 mg/mL

NADPH (cofactor) 1 mM

Potassium Phosphate, pH 7.4 100 mM

Magnesium Chloride 5 mM

### APPENDIX 1. ANALYTICAL METHOD

Liquid Chromatography

Column: Thermo BDS Hypersil C18 30×2.1 mm, 3 μm, with guard column

M.P. Buffer: 25 mM ammonium formate buffer, pH 3.5

Aqueous Reservoir (A): 90% water, 10% buffer

Organic Reservoir (B): 90% acetonitrile, 10% buffer

Flow Rate: 700 μL/minute

Gradient Program:

| Time (min) | % A | % B |
|---|---|---|
| 0.0 | 100 | 0 |
| 1.0 | 0 | 100 |
| 1.5 | 0 | 100 |
| 1.6 | 100 | 0 |
| 2.5 | 100 | 0 |

Total Run Time: 2.5 minutes

Autosampler: 1-5 μL injection volume

Autosampler Wash: water/methanol/2-propanol:1/1/1; with 0.2% formic acid

Mass Spectrometer

Instrument: PE SCIEX API 4000

Interface: Turbo Ionspray

Mode: Multiple reaction monitoring

Method: 2.5 minute duration

### Test Article 1 μM

| | | % Remaining of Initial (n = 1) | | | | | Half-life^{a} | CLᵢₙₜ^{b} (mL/min/ |
|---|---|---|---|---|---|---|---|---|
| Test Article | Species | 0 min | 10 min | 20 min | 30 min | 60 min | (min) | mg protein) |
| EZ1 HCl | Human | 100 | 94.9 | 95.5 | 93.7 | 95.0 | >60 | <0.0231 |
| EZ-II-75 | Human | 100 | 96.4 | 101 | 98.8 | 103 | >60 | <0.0231 |

Note (a): When the calculated half-life is longer than the duration of the experiment, the half-life is expressed as being greater than the longest incubation time. Note (b): Intrinsic clearance (CLᵢₙₜ) was calculated based on CLᵢₙₜ=k/P, where k is the elimination constant and P is the protein concentration in the incubation. Testosterone was used as a test compound and had a half-life of 40.6 minutes and a CLᵢₙₜ of 0.0341 mL/min/mg protein. The tested compounds, EZ1 HCl and EZ-II-75, exhibited excellent stability in human liver microsomes.

### Example 3—Enzymatic Activity

Various compounds were tested for enzymatic activity with respect to Lys 18 and Lys 27 of histone 3. Results are provided in below in the following Table. EC50 is indicated for activators and IC50 for inhibitors respectively.

| | H3-Lys 27 | | H3-Lys 18 | |
|---|---|---|---|---|
| COMPOUND | EC50 | IC50 | EC50 | IC50 |
| JF2 IN DMSO (n = 5) | NO ACTIVITY | NO ACTIVITY | 137.99 nM ± 0.37 | — |
| JF17 in DMSO (n = 5) | TO BE DETERMINED | TO BE DETERMINED | 102.83 nM ± 0.83 | — |
| JF19 in DMSO (n = 5) | NO ACTIVITY | NO ACTIVITY | NO ACTIVITY | NO ACTIVITY |
| EZ1 HCl in DMSO | NO ACTIVITY | NO ACTIVITY | 97.04 nM ± 0.44 | — |
| (n = 6) | | | | |
| EZ II-75 in DMSO | NO ACTIVITY | NO ACTIVITY | NO ACTIVITY | NO ACTIVITY |
| (n = 6) | | | | |

### Example 4: Efficacy of YF2

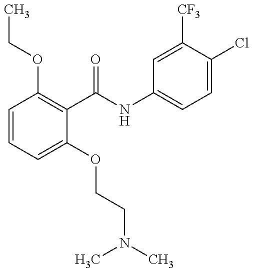 [SMILES: CCOc1cccc(OCCN(C)C)c1C(=O)Nc1ccc(Cl)c(C(F)(F)F)c1]

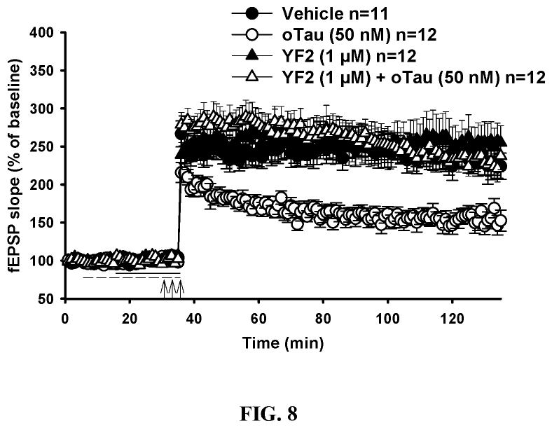

against tau induced synaptic dysfunction. Oligomeric tau caused a defect of LTP. YF2 was applied to hippocampal slices either alone or concurrently with oligomeric tau. The compound rescued the oligomeric tau induced defect in long-term potentiation. FIG. 8 is a graph illustrating that YF2 rescues oTau-induced LTP deficits. LTP was impaired in hippocampal slices from WT mice perfused with oTau. The concurrent treatment with YF2 and oTau reestablished normal LTP. There was no impairment in slices treated with YF2 or vehicle. The horizontal solid bar represents oTau perfusion while the horizontal dashed bar represents drug treatment. The three arrows correspond to the theta-burst stimulation. Two-Way ANOVA Vehicle vs. oTau: F(1, 21)=21.32, p=0.0001; Vehicle vs. YF2: F(1, 21)=0.200, p=0.6593; Vehicle vs. YF2+oTau: F(1, 22)=0.3455, p=0.563; YF2 vs. oTau: F(1, 22)=13.72, p=0.0012; YF2 vs. YF2+oTau: F(1, 23)=0.00043, p=0.984; YF2+oTau vs. oTau: F(1, 23)=31.52, p<0.0001.

### Example 5: Efficacy of YF2 Against Tau Induced Memory Loss

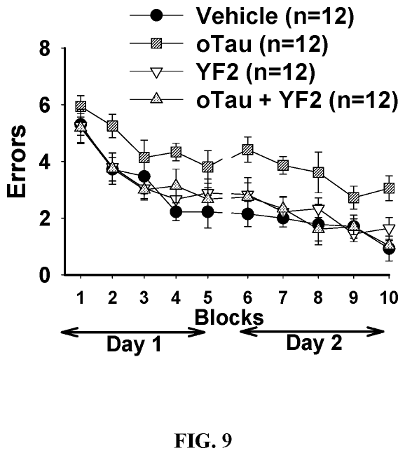

Oligomeric tau caused a defect of spatial memory and associative memory. YF2 was injected in mice either alone or concurrently with oligomeric tau. The compound rescued the oligomeric tau induced defect in memory. FIG. 9 illustrates that YF2 rescues oTau-induced defects in the 2 day radial arm water maze test of spatial short-term memory. The performance in the RAWM was impaired in mice administered with oTau (500 nM). Treatment with the HAT activator YF2 (5 mg/kg) rescued the deficit. The performance was not impaired when mice were treated with only YF2 or vehicle. (ANOVA for repeated measures among all groups at day 2: F(3, 47)=10.27, p<0.0001. One-way ANOVA for block 10: F(3, 47)=9.496, p<0.0001; Bonferroni's p<0.0001 oTau vs. vehicle and oTau vs. YF2 alone. p=0.025 for oTau+YF2 vs. oTau. YF2 alone did not modify memory (Bonferroni's p=1 compared to vehicle, block 10).

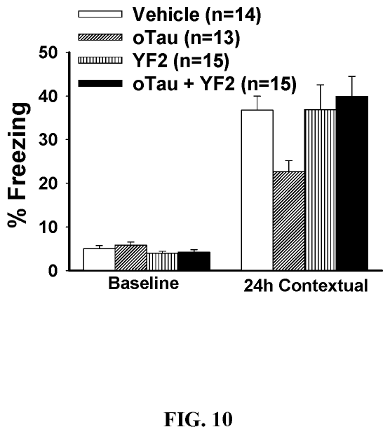

FIG. 10 shows that YF2 rescues oTau-induced deficit in contextual fear memory. A statistical significant difference is visible when comparing all groups during testing for contextual fear memory at 24 hrs after the electric shock (ANOVA among all groups: F(3, 53)=3.051 p=0.0364). Comparisons between groups revealed a statistically significant difference in freezing behavior when comparing mice that received YF2 plus oTau with oTau-administered animals (t-test: t(26)=3.13, p<0.05). Furthermore, oTau-treated mice showed amounts of freezing which were statistically different from vehicle-treated mice (t-test: t(25)=3.158, p<0.05), and YF2 alone did not affect memory compared to vehicle-treated mice (t-test: t(25)=0.015, p>0.05). There were no differences in the baseline freezing between groups (ANOVA: F(3,53)=2.107 p=0.1103).

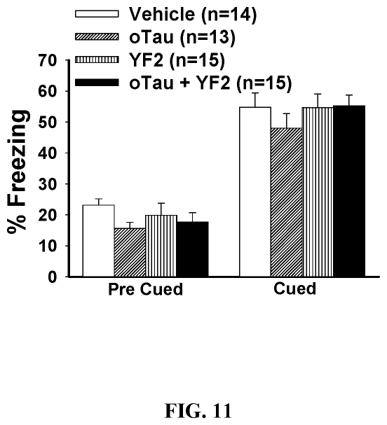

FIG. 11 shows that the average freezing in cued fear associative memory test in the presence oTau is not affected by YF2. No difference was detected between groups in freezing behavior before (pre cue, ANOVA: F(3, 53)=1.16, p=0.335) and after (post cue, ANOVA: F(3, 53)=0.599, p=0.6187) the auditory cue in the cued conditioning test.

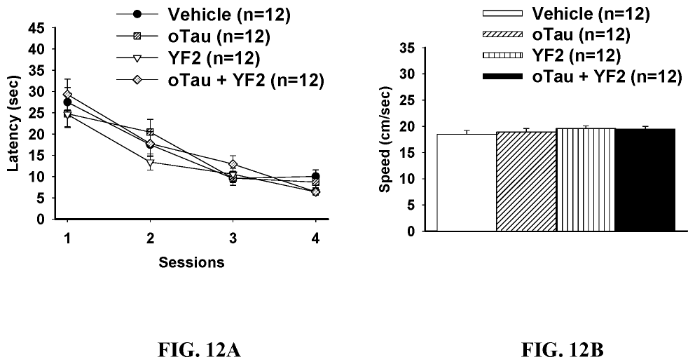

FIGS. 12A and 12B provide graphs showing that during visible platform test, the average time and speed to reach a platform located above the surface of the water in the presence oTau is not affected by YF2. Visible platform testing to evaluate motivational, visual and motor deficits showed no differences between groups in latency to reach the platform (ANOVA for repeated measures: F(3, 47)=0.691, p=0.6041) (A) and swimming speed (ANOVA: F(3, 47)=0.409, p=0.747) (B).

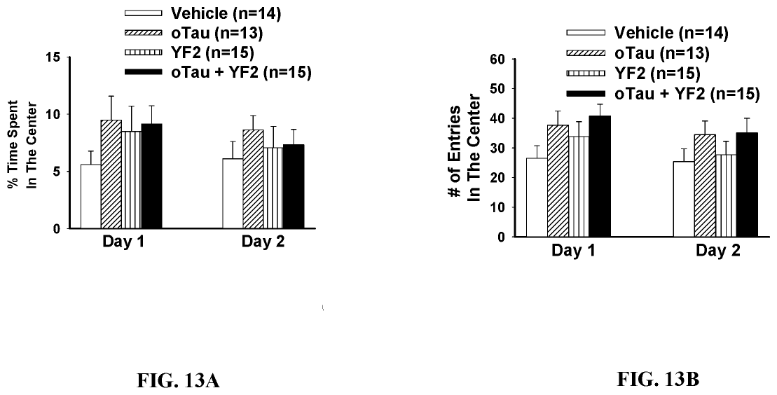

FIG. 13A and FIG. 13B show that during Open Field test, the time spent in the center of the arena and the number of entries in the center in the presence oTau are not affected by YF2. FIG. 13A—No differences were observed in the time spent in the center compartment (ANOVA: F(3, 53)=0.431, p=0.7313) and FIG. 13B—number of entries into the center compartment (ANOVA: F(3, 53)=1.094, p=0.3596) between groups on the second day in the open field test.

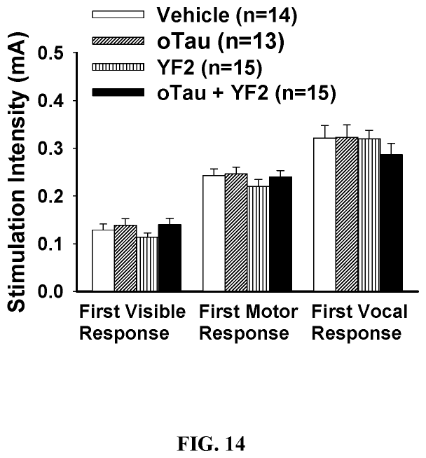

FIG. 14 is a graph showing that during sensory threshold assessment, animal capability to perceive the electric shock in the presence oTau is not affected by YF2. There were no statistically significant differences among groups during the assessment of the sensory threshold (ANOVA among all groups: visible response F(3, 53)=1.039, p=0.3831; motor response F(3, 53)=0.728, p=0.540 and audible response F(3, 53)=0.5812, p=0.6299).

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and/or rearranged in various ways within the scope and spirit of the invention to produce further embodiments that are also within the scope of the invention. Those skilled in the art will recognize, or be able to ascertain, using no more than routine experimentation, numerous equivalents to the specific embodiments described specifically in this disclosure. Such equivalents are intended to be encompassed in the scope of the following claims.

1. A compound of formula (1), 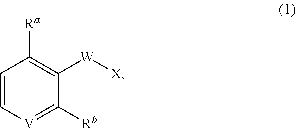
wherein
X is alkyl, 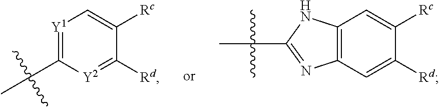
V, Y¹ and Y² are independently —CH— or —N—;
W is —CH₂N(R^{e})—, or —C(O)N(R^{f})—; or
W and R^{b} together with the atoms to which they are bound form a structure of formula Z, where Z is 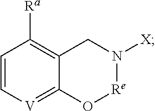
R^{a} is —H, —OH, —O—(C₁-C₆)-alkyl, —O—(C₂-C₆)-heteroalkyl or —O—(C₂-C₆)—N(R^{f}R^{g});
R^{b} is —H, -halo, —(C₂-C₆)-heteroalkyl, —OH, —O—(C₁-C₆)-alkyl, —O—(C₂-C₆)-heteroalkyl, —N—(C₂-C₆)-heteroalkyl, —O—(C₂-C₆)—O-alkyl, —O—(C₂-C₆)—N(R^{f}R^{g}) or N(R^{f})—(C₂-C₆)—N(R^{f}R^{g});
R^{c} and R^{d} are independently —H, -halo or -haloalkyl;
R^{e} is —CH₂— or —C(O)—; and
R^{f} and R^{g} are independently —H, —(C₁-C₆)-alkyl or —(C₂-C₆)-heteroalkyl;
wherein
a) Y¹ and Y² are both —N—; or
b) only one of Y¹ and Y² is —N— and when X is 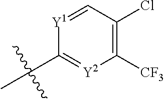
and R^{b} is —O(CH₂)₂—N(CH₃)₂, then R^{a} is —H, —OH, —O-methyl, O—(C₃-C₆)-alkyl or —O—(C₂-C₆)—N(C₁-C₆-alkyl)₂; or
c) W and R^{b} together with the atoms to which they are bound form the structure of formula Z, where Z is 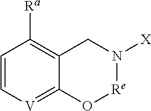
and when X is 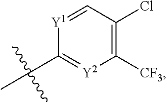
then R^{a} is —H, —OH, —O-methyl, O—(C₃-C₆)-alkyl or —O—(C₂-C₆)—N(C₁-C₆-alkyl)₂;
or a pharmaceutically acceptable salt thereof.

2. The compound of claim 1, wherein the compound is 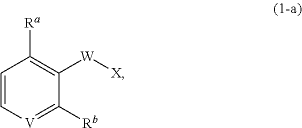
wherein
X is alkyl, 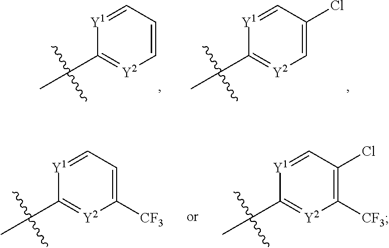
V, Y¹ and Y² are independently —CH— and —N—;
W is —C(O)N(R^{f})—;
R^{a} is —H, —OH, —O—(C₁-C₆)-alkyl, —O—(C₂-C₆)-heteroalkyl or —O—(C₂-C₆)—N(R^{f}R^{g});
R^{b} is —H, -halo, —(C₂-C₆)-heteroalkyl, —OH, —O—(C₁-C₆)-alkyl, —O—(C₂-C₆)-heteroalkyl, —N—(C₂-C₆)-heteroalkyl, —O—(C₂-C₆)—O-alkyl, —O—(C₂-C₆)—N(R^{f}R^{g}) or N(R^{f})—(C₂-C₆)—N(R^{f}R^{g}); and
R^{f} and R^{g} are independently —H, —(C₁-C₆)-alkyl or —(C₂-C₆)-heteroalkyl; or a pharmaceutically acceptable salt thereof.

3. The compound of claim 1, wherein the compound is, 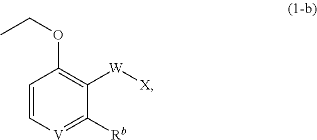
wherein
X is 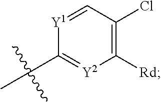
V, Y¹ and Y² are independently —CH— or —N—, wherein at least one of Y¹ and Y² is —N—;
W is —C(O)N(R^{f})—;
R^{b} is -halo, —O—(C₁-C₂)-alkyl, —O—(C₄-C₆)-alkyl, —O—(C₂-C₆)—OH, —O—(C₂-C₆)—O—(C₁-C₆)-alkyl, —O—(C₂-C₆)—NH₂, —O—(C₂-C₆)—NH(C₁-C₆-alkyl), —O—(C₄-C₆)—N(C₁-C₆-alkyl)₂ or —N(C₁-C₆-alkyl)-(C₂-C₆)-alkyl-N(C₁-C₆-alkyl)₂; and
R^{f} is —H or —(C₂-C₆)-alkyl-N(C₁-C₆-alkyl)₂; or a pharmaceutically acceptable salt thereof.

4. The compound of claim 1, wherein the compound is, 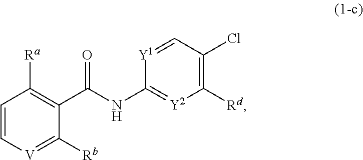
wherein
V, Y¹ and Y² are independently —CH— or —N—;
R^{a} is —H, —OH, —O-methyl, O—(C₃-C₆)-alkyl or —O—(C₂-C₆)—N(C₁-C₆-alkyl)₂; and
R^{b} is halo, —OH, —O—(C₁-C₆)-alkyl or —O—(C₃-C₆)—N(C₁-C₆-alkyl)₂ or —N(R^{f})—(C₃-C₆)-alkyl-N(C₁-C₆-alkyl); and
R^{f} is independently —H, —(C₁-C₆)-alkyl or —(C₂-C₆)-heteroalkyl; or a pharmaceutically acceptable salt thereof.

5. The compound of claim 1, wherein the compound is 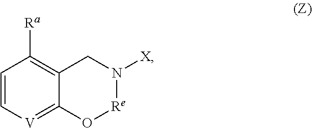
wherein
X is 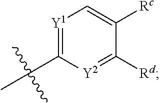
V, Y¹ and Y² are independently —CH— or —N—;
R^{a} is —H, —OH, —O-methyl, —O—(C₃-C₆)-alkyl, —O—(C₂-C₆)-heteroalkyl or —O—(C₂-C₆)—N(R^{f}R^{g});
R^{c} and R^{d} are independently —H, -halo or -haloalkyl; and
R^{e} is —CH₂— or —C(O)—; and
R^{f} and R^{g} are independently —H, —(C₁-C₆)-alkyl or —(C₂-C₆)-heteroalkyl; or a pharmaceutically acceptable salt thereof.

6. The compound of claim 1, wherein the compound is 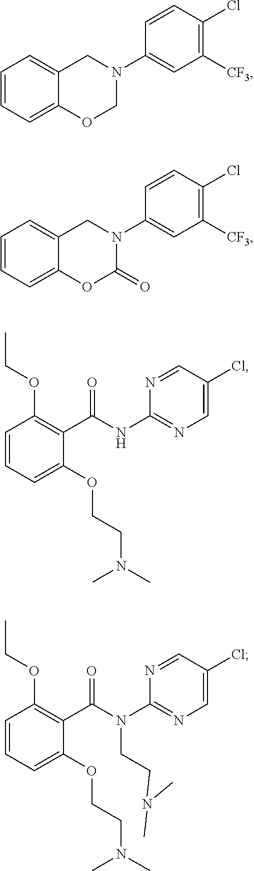 [SMILES: FC(F)(F)c1cc(N2COc3ccccc3C2)ccc1Cl ; O=C1Oc2ccccc2CN1c1ccc(Cl)c(C(F)(F)F)c1 ; CCOc1cccc(OCCN(C)C)c1C(=O)Nc1ncc(Cl)cn1 ; CCOc1cccc(OCCN(C)C)c1C(=O)N(CCN(C)C)c1ncc(Cl)cn1]
or a pharmaceutically acceptable salt thereof.

7. The compound of claim 1, wherein the compound regulates HAT activity.

8. The compound of claim 7, wherein the compound is a HAT activator.

9. The compound of claim 7, wherein the compound is a HAT inhibitor.

10. The compound of claim 8, wherein the HAT activator is 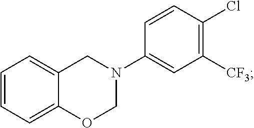 [SMILES: FC(F)(F)c1cc(N2COc3ccccc3C2)ccc1Cl]
or a pharmaceutically acceptable salt thereof.

11. A pharmaceutical composition comprising a compound of claim 1.

12. A method of treating a neurodegenerative disease in a subject in need thereof, comprising administering to said subject therapeutically effective amount of a compound of claim 1.

13. The method of claim 12, wherein the neurodegenerative disease comprises adrenoleukodystrophy (ALD), Alexander's disease, Alpers' disease, Alzheimer's disease, corticobasal degeneration (CBD), argyrophilic grain disease (AGD), and globular glial tauopathy (GGT), the neurofibrillary tangle-predominant senile dementia (now included also in the category of primary age-related tauopathy, PART), behavioral variant frontotemporal dementia; Semantic variant primary progressive aphasia, non-fluent/agrammatic variant primary progressive aphasia, logopenic variant primary progressive aphasia, Rubinstein-Taybi syndrome, amyotrophic lateral sclerosis (Lou Gehrig's disease), ataxia telangiectasia, batten disease (also known as Spielmeyer-Vogt-Sjögren-Batten disease), bovine spongiform encephalopathy (BSE), canavan disease, cockayne syndrome, corticobasal degeneration, Creutzfeldt-Jakob disease, familial fatal insomnia, frontotemporal lobar degeneration, Huntington's disease, HIV-associated dementia, Kennedy's disease, Krabbe's disease, Lewy body dementia, neuroborreliosis, Machado-Joseph disease (spinocerebellar ataxia type 3), multiple system atrophy, multiple sclerosis, narcolepsy, Niemann Pick disease, Parkinson's disease, Pelizaeus-Merzbacher disease, Pick's disease, primary lateral sclerosis, Prion diseases, progressive supranuclear palsy, Refsum's disease, Sandhoff disease, Schilder's disease, subacute combined degeneration of spinal cord secondary to pernicious anemia, Spielmeyer-Vogt-Sjogren-Batten disease (also known as Batten disease), spinocerebellar ataxia (multiple types with varying characteristics), spinal muscular atrophy, Steele-Richardson-Olszewski disease, Tabes dorsalis or toxic encephalopathy.

14. The method of claim 13, wherein the neurodegenerative disease comprises Alzheimer's disease, Parkinson's disease or Huntington's disease.

15. A method of treating cancer in a subject in need thereof, comprising administering to the subject a therapeutically effective amount of a compound of claim 1.

16. The method of claim 15, wherein the cancer comprises B cell lymphoma, diffuse large B-cell lymphoma (DLBCL), germinal center-derived DLBCL, activated B-cell-derived (ABC) DLBCL, non-germinal center DLBCL, colon cancer, lung cancer, non-small cell lung cancer (SCLC), renal cancer, bladder cancer, peripheral T cell lymphoma (PTCL-NOS), NK/T cell lymphoma (NKTCL), follicular lymphoma, myeloma, leukemia, chronic myeloid leukemia, acute myeloid leukemia, chronic lymphocytic leukemia, acute lymphocytic leukemia, hematopoietic neoplasias, thymoma, lymphoma, sarcoma, lung cancer, liver cancer, non-Hodgkin's lymphoma, Hodgkin's lymphoma, uterine cancer, renal cell carcinoma, hepatoma, adenocarcinoma, breast cancer, pancreatic cancer, liver cancer, prostate cancer, head and neck carcinoma, thyroid carcinoma, soft tissue sarcoma, ovarian cancer, primary or metastatic melanoma, squamous cell carcinoma, basal cell carcinoma, brain cancer, angiosarcoma, hemangiosarcoma, bone sarcoma, fibrosarcoma, myxosarcoma, liposarcoma, chondrosarcoma, osteogenic sarcoma, chordoma, angiosarcoma, endotheliosarcoma, lymphangiosarcoma, lymphangioendothelioma, synovioma, testicular cancer, uterine cancer, cervical cancer, gastrointestinal cancer, stomach cancer, esophageal cancer, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, Waldenstrom's macroglobulinemia, papillary adenocarcinomas, cystadenocarcinoma, bronchogenic carcinoma, bile duct carcinoma, choriocarcinoma, seminoma, embryonal carcinoma, Wilms' tumor, lung carcinoma, epithelial carcinoma, cervical cancer, testicular tumor, glioma, astrocytoma, medulloblastoma, craniopharyngioma, ependymoma, pinealoma, hemangioblastoma, acoustic neuroma, oligodendroglioma, meningioma, retinoblastoma, leukemia, melanoma, neuroblastoma, small cell lung carcinoma, bladder carcinoma, lymphoma, multiple myeloma or medullary carcinoma.

17. The method of claim 14, wherein the method comprises preventing, restoring or otherwise improving motor skills, learning, memory or cognition.

18. A method of improving memory in a subject not suffering from a neurodegenerative disease, comprising administering to said subject a therapeutically effective amount of the compound of claim 1.

19. A method of treating arteriogenesis, Kawasaki disease, Crohn's disease and other inflammatory conditions, DiGeorge syndrome, Rubenstein-Taybi syndrome (RTS), cardiac hypertrophy, insulin resistance, diabetes, type 2 diabetes, obesity, lymphoid hyperplasia or chronic kidney disease in a subject in need thereof, comprising administering to said subject a therapeutically effective amount of a compound of claim 1.

20. The method of claim 12, wherein the subject has at least one mutant HAT enzyme gene.

21. The method of claim 20, wherein a mutation in at least one allele of either the EP300 or CREBBP genes is present in the subject.

22. The method of claim 21, wherein a mutant EP300 gene is present in the subject.

23. The method of claim 12, wherein a HDAC regulator is co-administered with said HAT activator or inhibitor.

24. The method of claim 23, wherein the HDAC regulator is a HDAC inhibitor.

25. The method of claim 24, wherein the HDAC inhibitor is romidepsin.

26. The method of claim 23, wherein the HDAC regulator and the HAT activator or inhibitor are administered at different times.
